(19) United States
(12) Patent Application Publication
(10) Pub. No.: US 2024/0391890 A1
(43) Pub. Date: Nov. 28, 2024

(54) PROCESS FOR THE PREPARATION OF A FURFURAL DERIVATIVE

(71) Applicant: Furanix Technologies B.V., Amsterdam (NL)

(72) Inventors: Ana Sofia Vagueiro De Sousa Dias, Amsterdam (NL); Jeffrey John Kolstad, Amsterdam (NL); Johannes Maria Franciscus Sijben, Amsterdam (NL); Jagdeep Singh, Amsterdam (NL)

(21) Appl. No.: 18/695,867

(22) PCT Filed: Sep. 16, 2022
(86) PCT No.: PCT/EP2022/075852

§ 371 Date: Mar. 27, 2024

(30) Foreign Application Priority Data
Oct. 21, 2021 (EP) 21203917.6

(51) Int. Cl. C07D 307/46

(52) U.S. Cl. CPC C07D 307/46

(57) ABSTRACT

A process for the preparation of a furfural derivative includes (i) reacting a fructose- and/or glucose-containing starting material with a liquid hydroxyl group-containing organic compound in the presence of an acid to produce an acid reaction mixture including the furfural derivative; (ii) neutralizing at least part of the acid reaction mixture obtained in step (i) to provide a partially neutralized reaction mixture, (iii) separating at least part of the partially neutralized reaction mixture obtained in step (ii) to obtain a stream depleted of furfural derivative and a stream including furfural derivative; (iv) contacting at least part of the depleted stream obtained in step (iii) with a solvent to obtain a mixture of the solvent and the depleted stream; and (v) separating at least part of the mixture obtained in step (iv) into solvent containing the furfural derivative and a stream further depleted of the furfural derivative.

### CROSS-REFERENCE TO RELATED APPLICATIONS

This application is the National Stage of International Application No. PCT/EP2022/075852, filed Sep. 16, 2022, which claims the benefit of European Application No. 21203917.6, filed Oct. 21, 2021, the contents of which is incorporated by reference herein.

### FIELD OF THE INVENTION

The present invention relates to a process for the preparation of a furfural derivative.

The furfural derivative can be described as having the chemical formula (1):

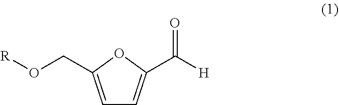

wherein R represents hydrogen, an alkyl or an acyl group.

### BACKGROUND OF THE INVENTION

Furfural derivatives of chemical formula (1), including 5-hydroxymethylfurfural (HMF), 5-alkoxymethylfurfural (AlkMF) and 5-acyloxymethylfurfural (AcMF) are interesting chemicals. The furfural derivatives find application as precursor for e.g. furan dicarboxylic acid, an important monomer for polyesters, polyamides and polyurethanes. HMF has further antibacterial and anticorrosive properties. HMF, AlkMF and AcMF can be derived from sustainable sources. The furfural derivatives may be derived from a variety of carbohydrates, in particular from hexoses, such as fructose and glucose. Raw materials such as starch, cellulose, sucrose or inulin can be used as starting products for the manufacture of hexoses.

HMF, AlkMF and AcMF can be obtained from sustainable sources such as the process described in U.S. Pat. No. 7,317,116 for the preparation of HMF. This process comprises heating a fructose source, such as high fructose corn syrup, and an organic solvent in the presence of an acid catalyst to achieve the acid-catalyzed dehydration reaction of fructose. The resulting product may then be neutralized to a pH of 7 to 9, e.g. by the gradual addition of sodium hydroxide. In examples the neutralization is carried out to pH values of at least 7.5. Subsequently, the thus neutralized product was subjected to distillation to remove the solvent.

WO 2107/003294 describes a further process for increasing the amount of desired product. This process comprises reacting a fructose- and/or glucose-containing starting material with a liquid hydroxyl-containing compound, separating at least part of the liquid hydroxyl-group containing compound from the primary reaction mixture to yield a secondary acid reaction mixture and subsequently neutralizing and purifying the product obtained. WO 2107/003294 does not describe or hint at further increasing the amount of desired product by treating side-streams of the process or at further removing compounds from side-streams.

The article “Autocatalytic Production of 5-Hydroxymethylfurfural from Fructose-Based Carbohydrates in a Biphasic System and Its Purification” by Hao Ma et al., Ind. Eng. Chem. Res. 2015, 54, 2657-2666 describes a process of converting fructose into 5-hydroxymethylfurfural (HMF) in a biphasic solvent system of methyl isobutyl ketone and water without any external catalyst. NaOH neutralization of the mixture obtained allowed to achieve a high recovery of HMF of high purity. The use of a protic organic solvent such as methanol, ethanol, n-propanol and n-butanol is described to give very low fructose conversion.

The article “Recovery and separation of carbohydrate derivatives from the lipid extracted alga Dunaliella by mild liquefaction” by Rikho-Strukman Liisa K. et al., ACS Sustainable Chemistry & Engineering, vol. 5, no. 1, 3 Jan. 2017, pages 588-595 describes HMF extraction from algae residue. It was found that 96.2% of HMF can be recovered in methyl iso butyl ketone (MIBK) rich extract.

The article “Extraction of furfural and furfural/5-hydroxymethylfurfural from mixed lignocellulosic biomass-derived feedstocks” by Wang Zhaoxing et al., ACS Sustainable Chemistry & Engineering, vol. 9, no. 22, 7 Jun. 2021, pages 7489-7498 describes single component (furfural) and mixture (furfural and HMF) partition coefficients in various solvents from lignocellulosic biomass dehydration products. The slight difference in molecular structure between furfural and HMF results in differences in separation.

The article “Liquid extraction of furfural from aqueous solution” by John R. Croker et al., Ind. Eng. Chem. Fundam. 1984, 23, pages 480-484 describes recovering furfural from aqueous solutions.

### SUMMARY OF THE INVENTION

The present invention concerns a process in which a fructose- and/or glucose-containing starting material is converted in the presence of a liquid hydroxyl group-containing organic solvent. The reaction mixture obtained tends to contain a wide variety of products besides furfural derivatives according to formula (1) and the liquid hydroxyl group-containing organic solvent namely water from the dehydration of the fructose and/or glucose and by-products of the acid catalyzed fructose and/or glucose conversion such as methyllevulinic acid, formic acid, furfural and angelica lactone. This mixture of compounds was found to be challenging to separate especially as azeotropic mixtures with water tend to be formed. An additional challenge is that the liquid hydroxyl group-containing organic compound tends to act as co-solvent thereby hampering separation into different fractions after solvent extraction and strongly reducing the efficiency of extraction.

An aim of the present invention was to ensure that waste water produced in acid catalyzed fructose and/or glucose conversion in the presence of liquid hydroxyl group-containing organic compound would meet stringent environmental requirements especially a strongly reduced amount of methoxymethylfurfural and/or methyl levulinate, and to achieve this faster, i.e. in less extraction steps. Although certain process steps were known per se, there was no teaching, hint or suggestion to combine process steps according to the present invention let alone that such combination allows to faster separate a complex mixture as obtained by acid catalyzed conversion of fructose and/or glucose.

A further aim can be to recover further useful compounds from waste streams produced in acid catalyzed conversion of fructose and/or glucose. This allows increased efficiency and production of desired products.

An additional aim can be to ensure that liquid hydroxyl group-containing organic compound is substantially removed without building up in an extraction solvent recycle.

An additional aim can be to ensure that formic acid is substantially removed without building up in an extraction solvent recycle.

Surprisingly, it was found possible to treat in a simple and efficient way waste water streams from the acid catalyzed conversion of fructose and/or glucose which treated waste water met stringent environmental requirements.

The process of the present invention is for the preparation of a furfural derivative having the chemical formula (1)

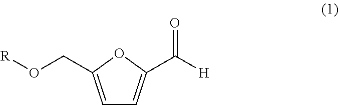

wherein R represents hydrogen, an alkyl group or an acyl group, which process comprises (i) reacting a fructose- and/or glucose-containing starting material with a liquid hydroxyl group-containing organic compound in the presence of an acid to produce an acid reaction mixture comprising the furfural derivative of chemical formula (1) and by-products; (ii) neutralizing at least part of the acid reaction mixture obtained in step (i) to a pH-value in the range of 3 to 6.5 to provide a partially neutralized reaction mixture, (iii) separating at least part of the partially neutralized reaction mixture obtained in step (ii) to obtain a stream depleted of furfural derivative of chemical formula (1) and a stream comprising furfural derivative of chemical formula (1); (iv) contacting at least part of the depleted stream obtained in step (iii) with a solvent selected from the group consisting of 4-methyl-2-pentanone, propyl acetate and toluene to obtain a mixture of the solvent and the depleted stream; and (v) separating at least part of the mixture obtained in step (iv) into solvent containing the furfural derivative of chemical formula (1) and/or by-products and a stream further depleted of the furfural derivative of chemical formula (1) and/or by-products

The many desired and undesired reaction products besides methanol, water and solvent make extraction and recycle build-up difficult to predict. Without wishing to be bound by any theory, it is thought that converting the by-product formic acid into its sodium salt reduces the amount of formic acid entrained in the extraction solvent thereby enhancing the recovery of the methoxymethylfurfural and/or methyl levulinate.

Furfural can be a side-product of the acid catalyzed conversion of fructose and/or glucose and tends to be difficult to remove as small amounts of water tend to already from azeotropes with furfural in solvent extraction. A further aim can be to limit the amount of furfural in solvent used in the process.

### BRIEF DESCRIPTION OF THE DRAWING

FIG. 1 shows a general concept of a process line-up of the present invention.

### DETAILED DESCRIPTION OF THE INVENTION

Process steps (iv) and (v) can be combined for example by using countercurrent solvent extraction.

The stream depleted of furfural derivative of chemical formula (1) obtained in step (iii) also is referred to as the depleted stream. The stream further depleted of the furfural derivative of chemical formula (1) and/or by-products obtained in step (v) also is referred to as further depleted stream. It will be clear that the process can comprise further steps before, after and between the various process steps. The depleted stream and the further depleted stream suitably are aqueous streams.

It will be clear to the skilled person that it can be advantageous to subject only part of a stream to a further process step. Generally, it is advantageous that at least part is at least 50% by weight (% wt), more preferably at least 90% wt, more preferably at least 95% wt.

The solvent for use in step (iv) is selected from the group consisting of 4-methyl-2-pentanone, propyl acetate and toluene. It was found that of 4-methyl-2-pentanone gave especially good results. 4-Methyl-2-pentanone is also referred to as methyl isobutyl ketone (MIBK) or isopropylacetone or isobutyl methyl ketone (IBMK). The person skilled in the art will be aware of additional compounds for further enhancing the solvent extraction of step (iii) and/or the subsequent phase separation of step (v). Preferably, the depleted stream is contacted solely with the solvent of the present invention. For a process with solvent recycle this means that solely solvent is added to make up for any loss of extraction solvent. Recycled solvent can contain additional compounds which are produced in the acid catalyzed conversion of fructose and/or glucose.

It was found that the presence of a substantial amount of liquid hydroxyl group-containing organic compound can hamper separation, more specifically phase separation, of the mixture of extraction solvent and depleted stream in step (v). Therefore, the depleted stream preferably comprises at most 10% by weight (% wt) of alcohol compounds, more preferably at most 8% wt, more preferably at most 5% wt, more preferably at most 4% wt, more preferably at most 3% wt, most preferably at most 2% wt, based on total amount of depleted stream. The alcohol compound for this aspect more specifically is an alkanol, more specifically an alkanol containing of from 1 to 3 carbon atoms, more specifically the total amount of methanol, ethanol and propanol. The propanol can be n-propanol or iso-propanol and preferably is n-propanol.

It was found that the volume ratio of extraction solvent to the depleted stream in step (iv) preferably is of from 0.1:1 to 1:1, preferably of from 0.20:1 to 0.80:1, more preferably of from 0.20:1 to 0.70:1.

The solvent and the depleted stream can be contacted in step (iv) in any way known to be suitable by the person skilled in the art. A preferred method is countercurrently contacting the solvent and the depleted stream.

The temperature of solvent extraction step (iii) is not critical. The solvent extraction preferably is carried out at of from ambient temperature to 100° C., more preferably of from ambient temperature to 50° C. The pressure has little influence on the reaction. Therefore, the pressure may vary between wide ranges at the discretion of the skilled person. The pressure preferably is of from ambient pressure up to 20 bar, more preferably up to 10 bar, more preferably up to 5 bar.

An especially preferred embodiment is contacting the depleted stream and the solvent in a counter current continuous extractor more preferably an asymmetrical rotating disc contactor column. It is possible to use any asymmetrical rotating disc contactor column known to be suitable to the person skilled in the art. An asymmetrical rotating disc contactor column is a cylindrical column which has a shaft at the center of the column. The column is divided into compartments by stator rings. A rotating disc is at the center of each compartment. The discs are mounted on the shaft. Fluid is mixed by rotation of the shaft. An asymmetrical rotating disc contactor thereby becomes a series of mixers and settlers which enhance mass transfer between the phases among the droplet interfacial area.

The depleted stream has the highest density and preferably enters an asymmetrical rotating disc contactor column at the top as continuous phase. Solvent enters from the bottom and preferably becomes a dispersed phase. Solvent tends to have the lowest density which will allow the solvent to trickle up thereby creating countercurrent flow.

Step (v) can be carried out in any way known to the person skilled in the art. Preferably, separation is carried out by allowing the mixture to phase separate.

Preferably, the process further comprises (vi) recovering furfural derivative of chemical formula (1) and/or by-products from the solvent containing the furfural derivative of chemical formula (1) and/or by-products to obtain a recovered solvent stream and a product mixture comprising furfural derivative of chemical formula (1) and/or by-products; and (vii) recycling at least part of the recovered solvent stream to step (iv). Furthermore, it is preferred that the process further comprises (viii) recovering furfural derivative of chemical formula (1) from the product mixture comprising furfural derivative of chemical formula (1) and/or by-products obtained in step (vi), preferably by evaporation or distillation.

It was found that the process steps (iv) and (v) allow to greatly reduce the amount of the furfural derivative of chemical formula (1) and by-products in waste streams especially aqueous waste streams. It was found possible to reduce the concentration of the furfural derivative of chemical formula (1) in the further depleted stream to 10% or less, more specifically 5% or less, more specifically 1% or less, of the concentration in the depleted stream. It was found to be possible to obtain a further depleted stream which could contain at most 100 (parts per million by weight (ppmw) of furfural derivative having the chemical formula (1), more specifically methoxymethylfurfural, more specifically at most 50 ppmw, more specifically at most 20 ppmw, more specifically at most 10 ppmw, more specifically at most 5 ppmw.

It was found that the present process allows to combine a limited build up of furfural in the extraction solvent while the furfural content of the further depleted stream can be brought to below environmentally acceptable discharge limits optionally after further treatment of the further depleted stream.

By-products obtained in step (i) can be any compound but specifically are selected from the group consisting of levulinic acid, formic acid, angelica lactone and esters of levulinic acid and formic acid.

The fructose- or glucose-containing starting material of step (i) may be selected from a variety of possible feedstocks. The starting material may comprise mono-, di-, oligo- or polysaccharides. The components of particular interest in biomass are those feedstocks that contain a monosaccharide or which can be readily processed to yield monosaccharides. Examples of suitable monosaccharides include fructose and mixtures of fructose with other monosaccharides, such as other hexoses and/or pentoses. Suitable other hexoses include but are not limited to glucose, galactose, psicose, mannose, and their oxidized derivatives, e.g. aldonic acid, reduced derivatives, e.g. alditol, etherified, esterified and amidated derivatives. The di- and oligosaccharide carbohydrates containing more than one saccharide unit, are suitably hydrolysed in the alcohol, resulting in a mixture of dissolved di- and/or oligosaccharides, monomeric saccharide units and/or glycoside units. Examples of suitable disaccharides include maltose, lactose, trehalose, turanose and sucrose, sucrose being preferred. Sucrose is abundantly available and therefore very suitable. The disaccharides can easily be converted into the monomeric units. Examples of suitable oligosaccharide are fructo-oligosaccharides which are found in many vegetables. By oligosaccharides is understood a carbohydrate that is built up of 3 to 10 monosaccharide units. Polysaccharides have more than ten monosaccharide units. These are polymeric structures formed of repeating units joined together by glycosidic bonds. The number of monosaccharide units in a polysaccharide may vary widely, and may range from 10 to 3000. Suitable polysaccharides include fructan, i.e. a polymer of fructose moieties, and levan, which is composed of D-fructofuranosyl moieties. Mixtures may also be used. Hydrolysis process streams from enzymatic or catalytic hydrolysis of starch, cellulose and hemi-cellulose or from alcoholysis processes that already contain mono- and disaccharides can suitably be used as starting material for the present process. In view of the above, the preferred monosaccharide is fructose, glucose and mixtures thereof. A suitable starting material is HFCS, i.e. high fructose corn syrup, comprising a major amount of fructose and some glucose. The preferred disaccharide is sucrose.

The fructose and/or glucose starting material may further comprise glycosides as described in WO 2012/091570.

The conditions under which the various steps of the present process can be carried out have been described in the prior art. The temperature for reacting a fructose- and/or glucose-containing starting material with a liquid hydroxyl group-containing organic compound in the presence of an acid catalyst preferably is in the range of 150 to 300° C., more preferably is of 175 to 225° C. The reaction time preferably is of from 1 minute to 10 minutes. The pressure is preferably in the range of 5 to 100 bar, more preferably from 10 to 60 bar. The process is preferably carried out as a continuous process.

It was found that it can be advantageous if the composition containing fructose- and/or glucose contains a limited amount of water besides the liquid hydroxyl-containing compound. It was found that the presence of water can increase the rate at which the fructose- and/or glucose dissolves besides the solubility of the fructose- and/or glucose. Therefore, the reaction mixture subjected to step (i) preferably contains water besides liquid hydroxyl-containing organic solvent. Such a proportion of water may suitably range from 0.5 to 20% wt, based on the weight of the liquid hydroxyl group-containing organic compound and water in the acid reaction mixture before start of the conversion of fructose- and/or glucose. The amount of water subsequently increases as a substantial amount of water is formed when fructose and/or glucose are converted during the reaction of step (i) to obtain the furfural derivatives of chemical formula (1).

The liquid hydroxyl-containing organic compound preferably contains from 1 to 6 carbon atoms, more preferably from 1 to 3 carbon atoms and preferably is an alkanol. Compounds containing a higher number of carbon atoms tend to become entrained in the solvent. Therefore, it is preferred that the liquid hydroxyl-containing compound is selected from the group consisting of methanol, ethanol and n-propanol, more specifically methanol and ethanol, most preferably is methanol.

The furfural derivative of chemical formula (1) is prepared from a reaction of a fructose- and/or glucose-containing starting material and liquid hydroxyl-containing compound in the presence of an acid catalyst. Suitable acid catalysts have been described in U.S. Pat. Nos. 7,317,116, 8,242,293 and 8,877,950. Such suitable catalysts include inorganic acids, such as sulfuric acid, phosphoric acid, hydrochloric acid and nitric acid, and organic acids, such as oxalic acid, levulinic acid, trifluoroacetic acid, methane sulfonic acid or p-toluene sulfonic acid. Immobilized acid catalysts in the form of e.g. sulfonic acid on resins may also be used. Other acid ion exchange resins are feasible as well as acid zeolites. Lewis acids, such as boron trifuoride or etherate complexes thereof, are further suitable catalysts. Also metals, such as Zn, Al, Cr, Ti, Th, Zr, and V can be used as catalyst in the form of ions, salts, or complexes. It appears that a wide range of acid components can be used as catalysts. The present process is very suitably carried out with an acid catalyst being a Brønsted acid selected from the group consisting of mineral inorganic acids, organic acids and mixtures thereof. Suitable mineral acids are sulfuric acid, nitric acid, hydrochloric acid and phosphoric acid, wherein sulfuric acid is particularly preferred. The organic acids are suitably selected from strong acids. Examples thereof include trifluoroacetic acid, methane sulfonic acid and p-toluene sulfonic acid.

The use of the mineral acids and strong organic acids suitably results in that the reaction mixture has a pH value of less than 3, and often even less than 2. However, it is also possible to arrive at low pH values when acid heterogeneous catalysts are used, such as acid ion exchange resins or acid zeolites. As indicated above, products of the conversion of fructose and/or glucose-containing starting materials may also include various organic acids such as levulinic acid and formic acid. The process of the present invention is therefore also suitable for embodiments wherein the reaction of the fructose and/or glucose-containing starting material is achieved with a heterogeneous, i.e. solid, catalyst. The acid reaction mixture preferably has a pH-value of less than 3.

At least part of the acid reaction mixture obtained in step (i) is neutralized to a pH-value in the range of 3 to 6.5 in step (ii) to provide a partially neutralized reaction mixture and at least part of such partially neutralized reaction mixture is used in step (iii) for recovering the stream comprising furfural derivative of chemical formula (1). Neutralization was found to improve the removal of furfural derivative of chemical formula (1) and/or by-products in step (iv).

The acid reaction mixture can be separated in step (iii) based on the volatility of the streams as the stream comprising furfural derivative tends to be the less volatile fraction while the depleted stream tends to be the more volatile fraction.

The stream containing furfural derivative of chemical formula (1) is preferably recovered from the acid reaction mixture in step (iii) by flash distillation or evaporation. The cut off point preferably will be between water and the liquid hydroxyl group-containing compound. If a mixture of liquid hydroxyl group-containing compounds is used, the person skilled in the art will be able to adjust the cut off point appropriately.

The furfural derivative with the chemical formula (1) can be recovered from the stream containing this compound obtained in step (iii) in any way known to be suitable to the skilled person. Preferably, the furfural derivative of chemical formula (1) is recovered by evaporation or distillation.

Suitably, at least part of the liquid hydroxyl group-containing organic compound is separated from the acid reaction mixture obtained in step (i) to provide a secondary acid reaction mixture which secondary acid reaction mixture is neutralized to a pH-value in the range of 3 to 6.5 to provide a partially neutralized secondary acid reaction mixture and subjecting at least part of the partially neutralized secondary acid reaction mixture to step (iii) for recovering a stream comprising the furfural derivative of chemical formula (1).

Preferably, the stream containing liquid hydroxyl group-containing compound which is separated from the acid reaction mixture is further separated into a water-rich fraction and a fraction rich in the liquid hydroxyl group-containing compound. This latter separation is preferably carried out by distillation. The water-rich fraction separated from the depleted stream preferably subsequently is subjected to step (iv).

It is advantageous to remove liquid hydroxyl group-containing compounds before any optional neutralization. Removal of at least part of the liquid hydroxyl group-containing compounds preferably includes the removal of water that has been formed during the reaction.

Furfural derivative of chemical formula (1) will be separated as part of the stream containing the furfural derivative. Furfural derivative can also be separated from other streams which contain furfural derivative. The furfural derivative may be obtained by evaporation or distillation. When the removal step is conducted as a distillation, also other products, such as levulinic acid, levulinate esters and formic acid, may be recovered. The distillation may be carried out in one or more columns as will be known to the skilled person. Wiped film evaporation was found to be especially suitable. Evaporation or distillation will result in a bottom residue. The bottom residue may comprise acid catalyst. It may further comprise salts if the acid reaction mixture has been subjected to neutralization.

Neutralization of acid reaction mixture is suitably accomplished by the addition of a base as neutralizing agent. The base can be selected from a variety of compounds. Such compounds include those that are disclosed in the above-mentioned U.S. Pat. No. 7,317,116, such as sodium hydroxide. However, the neutralizing agent may be selected from other bases, too. The acid reaction mixture is suitably neutralized by the addition of an alkali metal or alkaline earth metal hydroxide. Other suitable bases include organic bases such as amines. Such amines include mono-, di- or trisubstituted amines. The amines may be aliphatic, cycloaliphatic or aromatic. The basic nitrogen atom of the amine may be included in the cycloaliphatic or aromatic compound or be present as an amino substituent. Suitable amines include mono-, di- and tri (C₁-C₄ alkyl) amines as rather simple amino compounds. Other suitable organic bases are salts of organic oxides, such as alkoxides. Suitable alkoxides comprise alkyl moieties having from 1 to 6 carbon atoms. Examples of such alkoxides include methoxide, ethoxide, propoxide, t-butoxide and n-hexoxide salts. The counterion is suitably selected from alkali metal and alkaline earth metal ions. However, also quaternary ammonium ions can be used. These quaternary ammonium ions may be selected from protonated amines, such as the above-mentioned amines, but also tetra-substituted quaternary ammonium ions can be used. In view of the simplicity, the ammonium ion NH₄⁺ is the preferred ammonium ion. By neutralizing the acid reaction mixture acids are turned into salts that are to be removed from the eventual product. These salts may therefore end up in the eventual residue. Such eventual residue may comprise humin polymers. As described in U.S. Pat. No. 7,317,116 the reaction of fructose not only leads to HMF and similar products but via a competing side reaction also to humin polymers. These humin polymers, also known as humins, form a dark colored solid by-product. The salts of the neutralized acids may be separated from the product mixture together with the humins. In view of economic considerations together with the fact that hydroxides and alkoxides are easily admixed with the acid reaction mixture, the acid reaction mixture may suitably be neutralized by the addition of an alkali metal or alkaline earth metal hydroxide or alkoxide.

Neutralization preferably is conducted to a pH value in the range of 3 to 6.5. Advantageously the neutralization is conducted to as low a pH as feasible. That implies that the pH is increased to such a low value that only a little amount of neutralizing agent is to be added to the acid reaction mixture and at the same time that the degradation of valuable products such as the furfural derivative of chemical formula (1), but also compounds such as levulinic acid and esters thereof, does not take place. It has been found that very good results are obtained when the pH is brought into the range of 3 to 6, preferably from 3 to 5, more preferably from 3 to 4.5. Such neutralization can suitably be achieved by adding an aqueous solution of the neutralizing agent to the acid reaction mixture. At the same time the skilled person will realize that it is advantageous when the neutralizing agent is added in a form as concentrated as feasible. When the neutralizing agent is added to the acid reaction mixture in the form of a concentrated solution the acid reaction solution is suitably agitated to accomplish a distribution of the neutralizing agent as quickly and as homogeneously as possible to avoid the occurrence of any side reaction between any of the products in the acid reaction mixture and the neutralizing agent.

The amount of neutralizing agent depends on the process conditions. If fructose and/or glucose are reacted in the presence of sulphuric acid, the amount of neutralizing agent will generally to neutralize of from 0.8 to 1.5 of one of both acid groups of sulphuric acid, more specifically of from 1 to 1.2.

The conditions for neutralization are not critical. The pressure has little influence on the reaction. Therefore, the pressure may vary between wide ranges at the discretion of the skilled person. Suitable pressures include those in the range of 0.1 to 40 bar. Also the temperature for the neutralization may be selected from a wide range and is suitably selected such that the at least part of the acid reaction mixture is neutralized at a temperature in the range of 25 to 150° C.

The acid reaction mixture tends to contain water either formed during the conversion of the fructose and/or glucose-containing starting material or supplied as part of the solvent for the fructose and/or glucose conversion. Water preferably is removed after step (i).

Any solids that are obtained in any of the process steps are suitably removed by filtration. The solids comprise in particular humins that are the result of side-reactions of the fructose and/or glucose-containing starting material. Other solids may comprise solid salts, e.g. the salts that result from the addition of the neutralizing agent to the acid reaction mixture. Such salts may suitably comprise the alkali metal and/or alkaline earth metal salts of inorganic acids, such as sulfates, phosphates, chlorides or nitrates, when an inorganic acid is used as acid catalyst. Also such metal salts of organic acids are possible, such as alkali metal and/or alkaline earth metal salts of oxalic, p-toluene sulfonic, methane sulfonic, and trifluoroacetic acid, when such acids have been employed as acid catalyst. When a heterogeneous catalyst has been used, solid salts of products such as levulinic acid or formic acid may be formed. Solids removal is suitably carried out by filtration of the acid reaction mixture. In this way the filtrate of the acid reaction mixture is a homogenous liquid which facilitates handling, such as stirring during the neutralization. The filtered solids, such as humins, are suitably washed with water to remove acid catalyst, if any. Washing can suitably be done with water. In an alternative embodiment, the solids are separated by means of centrifugation.

Alternatively, the solids removal, e.g. filtration or centrifugation, is carried out after neutralization at the partially neutralized reaction mixture. Due to the neutralization, some solid salts may have been formed. Such salts are then suitably removed together with the humins fraction. If desired, the partially neutralized reaction mixture may be subjected to an evaporation step to remove at least some of the water and, optionally, alcohol or other volatile components, in order to concentrate the products and the solids so that most, if not all, of the salts formed are precipitated and removed together with the humins.

When the furfural derivative of chemical formula (1) is recovered from a process stream by evaporation or distillation, any bottom residue may comprise acid catalyst and/or dissolved salts resulting from the neutralization. Bottom residue is suitably treated to remove as much acid catalyst and as much salt as possible so that the remaining treated residue can be either combusted or discharged in another environmentally-friendly way. The use of alkaline earth hydroxides for neutralization is particularly preferred if the bottom residue is to be combusted to generate steam or other heating medium which can be used in the process to reduce the energy consumption. Most preferably, the alkaline earth compound is calcium.

Preferably, liquid hydroxyl group-containing organic compound which is separated off can be recycled back to step (i).

Preferably, further aqueous streams containing furfural derivative of chemical formula (1) and/or by-products which are obtained in other process steps also are treated in step (iv).

Furfural derivative of chemical formula (1) and/or by-products can be recovered from the solvent stream containing these compounds to obtain a stream comprising furfural derivative of chemical formula (1) and/or by-products. The stream comprising solvent which is thus obtained can be recycled to step (iv).

Entrained solvent can be removed from the further depleted stream and be recycled back to step (iv). The thus treated further depleted stream tends to contain a very strongly reduced amount of organic compounds. Such strongly reduced amount of organic compounds greatly reduces the burden on waste water facilities.

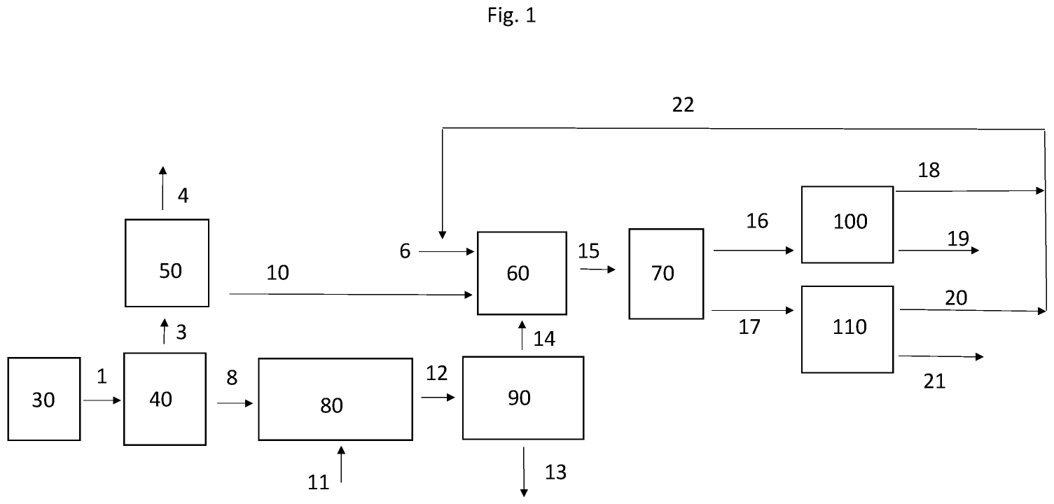

FIG. 1 shows a general concept of a process line-up of the present invention.

In the process of FIG. 1, a fructose- and/or glucose-containing starting material is reacted in unit 30 with methanol and a limited amount of water in the presence of an acid catalyst to produce an acid reaction mixture 1 comprising the furfural derivative of chemical formula (1) and by-products. Acid reaction mixture 1 is sent to a unit 40 to separate a more volatile fraction containing water and methanol and a less volatile fraction. The more volatile fraction is sent via line 3 to separation unit 50 where methanol is separated off and removed via line 4. This methanol can be recycled back to unit 30 if desired. After methanol has been removed, the remaining water-rich composition can be sent via line 10 to unit 60 to be treated together with other streams. The less volatile fraction obtained in unit 40 can be sent via line 8 to neutralization unit 80 to which base is added via line 11. The neutralized composition can be sent via line 12 to unit 90 in which a furfural derivative containing composition is separated and removed via line 13. This composition can be treated in subsequent steps not shown to further recover specific compounds. The depleted stream 14 is sent to extraction unit 60 in which depleted stream 14 is contacted with solvent from solvent stream 6. The mixture obtained is sent via line 15 to phase separation unit 70 in which the mixture is separated into solvent containing furfural derivative and/or by-products which is removed via line 16 and a stream further depleted of furfural and/or by-products which is removed via line 17. It will be clear to the person skilled in the art that an alternative is the use of a liquid-liquid extraction column to replace both units 60 and 70. Furfural derivative of chemical formula (1) and/or by-products can be removed from the solvent stream of line 16 in unit 100 to obtain a stream 18 comprising solvent and a stream 19 comprising furfural derivative of chemical formula (1) and/or by-products. The further depleted stream of line 17 can be separated in unit 110 into a solvent stream 20 and an aqueous waste stream 21. Solvent containing streams 18 and 20 can be recycled to unit 60 via line 22.

The present invention will be illustrated by means of the following examples.

### EXAMPLES

### Example 1

A feed composition as described in Table 1 was prepared to mimic product obtained by conversion of fructose reacted with methanol in the presence of sulfuric acid as a homogenous acid catalyst.

This composition was neutralized with concentrated sodium hydroxide and subsequently subjected to extraction with 31.83 grams of of 4-methyl-2-pentanone. The aqueous phase obtained after 1, 2 and 4 extraction steps was analyzed and the results are shown in Table 2 below.

Separately, a composition as described in Table 1 was subjected to extraction with the same amount of 4-methyl-2-pentanone without previous neutralization. Again, the aqueous phase obtained after 1, 2 and 4 extraction steps was analyzed and the results are shown in Table 2.

TABLE 1

| Compound | Weight (g) |
|---|---|
| Methoxymethylfurfural (MMF) | 5.819 |
| Methyl levulinate (ML) | 20.331 |
| Angelic lactone | 0.560 |
| Formic acid | 7.853 |
| Alpha-angelica lactone | 1.130 |
| Furfural | 1.524 |
| Methanol | 1.671 |
| Water | 118.153 |

TABLE 2

| | | Concentration of | Concentration of |
|---|---|---|---|
| | Number of | methoxymethylfurfural | methyl levulinate |
| | extractions | (MMF) (% wt/wt) | (ML) (% wt/wt) |
| With | 0 | 0.4 | 1.4 |
| neutralization | 1 | 0.05 | 0.4 |
| | 2 | | 0.2 |
| | 4 | | 0.05 |
| Without | 0 | 0.4 | 1.3 |
| neutralization | 1 | 0.1 | 0.6 |
| | 2 | 0.05 | 0.4 |
| | 4 | | 0.1 |

The above shows that neutralization followed by solvent extraction allows to reduce the amount of methoxymethylfurfural and/or methyl levulinate faster, i.e. in less extraction steps, of waste water stream produced in the conversion of fructose with methanol in the presence of sulfuric acid.

### Example 2

A fructose-containing starting material was reacted with methanol in the presence of sulfuric acid. Concentrated sodium hydroxide was added to neutralize the reaction mixture. To mimic commercial operation, the pH subsequently was lowered to a pH of 8 and additional methyl levulinate, methoxymethylfurfural and hydroxymethylfurfural were added. The composition which was subjected to extraction is shown in below Table 3.

TABLE 3

| | Weight amount on total |
|---|---|
| Compound | composition (% wt) |
| Methoxymethylfurfural (MMF) | 0.83 |
| Methyl levulinate (ML) | 1.69 |
| Hydroxymethylfurfural (HMF) | 0.04 |
| Levulinic acid salt | 0.41 |
| Formic acid salt | 0.69 |
| Alpha-angelica lactone | 0.05 |
| Unknown compounds | 2.63 |
| Methanol | 0.56 |
| Water | 93.1 |

Samples of about 100 g of the above composition were washed at 40° C. with 4-methyl-2-pentanone at different solvent to feed ratios at 200 revolutions per minute for 40 minutes. The amounts of methoxymethylfurfural (MMF), methyl levulinate (ML) and hydroxymethylfurfural (HMF) in the aqueous phase are shown in Table 4 below.

TABLE 4

| Solvent to | | ML | | MMF | | HMF |
|---|---|---|---|---|---|---|
| feed ratio | ML, % wt | efficiency | MMF, % wt | efficiency | HMF, % wt | efficiency |
| 0.50:1 | 0.03 | 98.4 | 0.00 | 99.6 | 0.01 | 74.9 |
| 0.33:1 | 0.10 | 92.7 | 0.01 | 98.7 | 0.02 | 48.2 |
| 0.25:1 | 0.27 | 84.5 | 0.05 | 96.8 | 0.03 | 40.7 |

The above shows that 4-methyl-2-pentanone allowed to prepare a waste water stream containing a strongly reduced amount of methoxymethylfurfural at different solvent to feed ratios.

### Example 3

Compositions comprising water and 26 mg/ml furfural and water and 36 mg/ml methyl levulinate were tested at room temperature. Of each of these compositions, a 1.5 ml sample was taken which was stirred for ten minutes. Subsequently, 1.5 solvent was added and the mixture was then again stirred for 10 minutes. Subsequently, the mixture was allowed to phase separate during one hour. Samples of 0.25 ml were taken of both the solvent phase and the aqueous phase. The methyl levulinate and furfural content were determined by gas chromatography. Based on these outcomes, the partition coefficient of the different solvents was calculated and is shown in Table 3. The partition coefficient is the ratio of the concentration of a specific compound in solvent to the concentration of the same compound furfural in water.

TABLE 5

| | Furfural partition | ML partition |
|---|---|---|
| Solvent | coefficient | coefficient |
| 1-Chloropentane | 3.1 | 1.2 |
| 4-Methyl-2-pentanone | 10.2 | 2.7 |
| Cyclohexane | 0.4 | 0 |
| Diisopropylether | 2 | 0.7 |
| Propyl acetate | 8.8 | 2.7 |
| Toluene | 4 | 1.8 |

The above shows that especially good furfural and methyl levulinate partition coefficients were obtained with the help of 4-methyl-2-pentanone, propyl acetate and toluene.

1. A process for the preparation of a furfural derivative having the chemical formula (1) 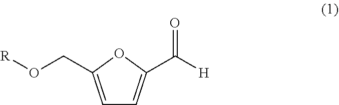
wherein R represents hydrogen, an alkyl group or an acyl group, which process comprises:
(i) reacting a fructose- and/or glucose-containing starting material with a liquid hydroxyl group-containing organic compound in the presence of an acid to produce an acid reaction mixture comprising the furfural derivative of chemical formula (1) and by-products;
(ii) neutralizing at least part of the acid reaction mixture obtained in step (i) to a pH-value in the range of 3 to 6.5 to provide a partially neutralized reaction mixture,
(iii) separating at least part of the partially neutralized reaction mixture obtained in step (ii) to obtain a stream depleted of furfural derivative of chemical formula (1) and a stream comprising furfural derivative of chemical formula (1);
(iv) contacting at least part of the depleted stream obtained in step (iii) with a solvent selected from the group consisting of 4-methyl-2-pentanone, propyl acetate and toluene to obtain a mixture of the solvent and the depleted stream; and
(v) separating at least part of the mixture obtained in step (iv) into solvent containing the furfural derivative of chemical formula (1) and/or by-products and a stream further depleted of the furfural derivative of chemical formula (1) and/or by-products.

2. The process according to claim 1, in which process at least part of the liquid hydroxyl group-containing organic compound is separated from the acid reaction mixture obtained in step (i) to provide a secondary acid reaction mixture which secondary acid reaction mixture is neutralized to a pH-value in the range of 3 to 6.5 to provide a partially neutralized secondary acid reaction mixture and subjecting at least part of the partially neutralized secondary acid reaction mixture to step (iii) for recovering a stream comprising the furfural derivative of chemical formula (1).

3. The process according to claim 1, wherein the liquid hydroxyl group-containing organic compound is selected from the group consisting of methanol, ethanol and n-propanol.

4. The process s according to claim 1 wherein the solvent is 4-methyl-2-pentanone.

5. The process according to claim 1 which process further comprises:
(vi) recovering furfural derivative of chemical formula (1) and/or by-products from the solvent containing the furfural derivative of chemical formula (1) and/or by-products to obtain a recovered solvent stream and a product mixture comprising furfural derivative of chemical formula (1) and/or by-products; and
(vii) recycling at least part of the recovered solvent stream to step (iv).

6. The process according to claim 5, which process further comprises:
(viii) recovering furfural derivative of chemical formula (1) from the product mixture comprising furfural derivative of chemical formula (1) and/or by-products obtained in step (vi), preferably by evaporation or distillation.

7. The process according to claim 1, wherein in step (iv) the volume ratio of the solvent to the depleted stream is of from 0.20:1 to 0.80:1, preferably of from 0.20:1 to 0.70:1.

8. The process according to claim 1, wherein the by-products are selected from the group consisting of furfural, levulinic acid, formic acid, angelica lactone and esters of levulinic acid and formic acid.

9. The process according to claim 1, wherein the stream comprising furfural derivative having the chemical formula (1) is recovered in step (iii) by evaporation or distillation.
